(19) United States
(12) Patent Application Publication
(10) Pub. No.: US 2023/0316517 A1
(43) Pub. Date: Oct. 5, 2023

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventors: Tsubasa GOTO, Tokyo (JP); Yoshiro Kitamura, Tokyo (JP)

(73) Assignee: FUJIFILM Corporation, Tokyo (JP)

(21) Appl. No.: 18/329,538

(22) Filed: Jun. 5, 2023

Related U.S. Application Data
(63) Continuation of application No. PCT/JP2021/041236, filed on Nov. 9, 2021.

(30) Foreign Application Priority Data
Dec. 22, 2020 (JP) 2020-212864

(51) Int. Cl. G06T 7/00; G06T 7/11; G06V 10/25; G06V 10/44; G06V 10/764; G16H 50/20

(52) U.S. Cl. CPC G06T 7/0012; G06T 7/11; G06V 10/25; G06V 10/44; G06V 10/764; G16H 50/20; G06T 2207/30061

(57) ABSTRACT

A processor is configured to: divide a target image into a plurality of first regions through a first division; divide the target image into a plurality of second regions through a second division different from the first division; derive a feature vector that represents at least a feature of each of the second regions for each of the first regions; derive a determination result for a target object included in the target image based on the feature vector; specify, among elements of the feature vector, an influential element that affects the determination result; and specify an influential region that affects the determination result in the target image based on the influential element.

### CROSS-REFERENCE TO RELATED APPLICATIONS

The present application is a Continuation of PCT International Application No. PCT/JP2021/041236, filed on Nov. 9, 2021, which claims priority to Japanese Patent Application No. 2020-212864, filed on Dec. 22, 2020. Each application above is hereby expressly incorporated by reference, in its entirety, into the present application.

### BACKGROUND

### Technical Field

The present disclosure relates to an information processing apparatus, an information processing method, and an information processing program.

### Related Art

In recent years, with the advancement of medical equipment, such as a computed tomography (CT) device and a magnetic resonance imaging (MRI) device, higher quality and high-resolution three-dimensional images have been used for image diagnosis.

Meanwhile, interstitial pneumonia and pneumonia caused by new coronavirus disease (coronavirus pneumonia) are known as lung diseases. In addition, a method of analyzing a CT image of a patient with interstitial pneumonia to classify and quantify tissues such as normal lung, blood vessels, and bronchus, as well as abnormalities such as honeycomb lung, reticular opacity, and ground-glass opacity included in the pulmonary field region of the CT image as properties has been proposed (see “Evaluation of computer-based computer tomography stratification against outcome models in connective tissue disease-related interstitial lung disease: a patient outcome study, Joseph Jacob 1, et al., BMC Medicine (2016) 14:190, DOI 10.1186/s12916-016-0739-7”, and “Quantitative evaluation of CT images of interstitial pneumonia by computer, Tae Iwasawa, Journal of the Japanese Association of Tomography, Vol. 41, No. 2, August 2014”. In this manner, by analyzing the CT image and classifying the properties to quantify the volume, the area, the number of pixels, and the like of the properties, it is possible to easily determine the degree of lung disease. As a method for classifying such properties, a model constructed by deep learning using a multi-layer neural network in which a plurality of processing layers are hierarchically connected has also been used (see JP2020-032043A).

Meanwhile, by using the classification results of the properties described above, it is also possible to determine whether or not to suffer from interstitial pneumonia, coronavirus pneumonia, or the like. However, in a case in which a physician determines a lung disease, which region in the lung a specific property is distributed in often affects the determination result of the disease.

### SUMMARY OF THE INVENTION

The present disclosure has been made in view of the above circumstances, and an object of the present disclosure is to make it possible to specify a region that affects a determination result for a target object.

According to the present disclosure, there is provided an information processing apparatus comprising: at least one processor, in which the processor is configured to:
divide a target image into a plurality of first regions through a first division;
divide the target image into a plurality of second regions through a second division different from the first division;
derive a feature vector that represents at least a feature of each of the second regions for each of the first regions;
derive a determination result for a target object included in the target image based on the feature vector;
specify, among elements of the feature vector, an influential element that affects the determination result; and
specify an influential region that affects the determination result in the target image based on the influential element.

In the information processing apparatus according to the present disclosure, the processor may be configured to enhance the influential region to display the target image.

In addition, in the information processing apparatus according to the present disclosure, the influential region may be at least one of a region of the target object, at least one region of the plurality of first regions, or at least one region of the plurality of second regions.

In addition, in the information processing apparatus according to the present disclosure, the target image may be a medical image, the target object may be an anatomical structure, and the determination result may be a determination result regarding presence or absence of a disease.

In addition, in the information processing apparatus according to the present disclosure, the first division may be a division based on a geometrical characteristic or an anatomical classification of the anatomical structure, and
the second division may be a division based on a property of the anatomical structure.

In addition, in the information processing apparatus according to the present disclosure, the processor may be configured to acquire the determination result for the target object by linearly discriminating each element of the feature vector.

In addition, in the information processing apparatus according to the present disclosure, the processor may be configured to perform the linear discrimination by comparing a weighted addition value of each element of the feature vector with a threshold value.

In addition, in the information processing apparatus according to the present disclosure, the processor may be configured to, in a case in which the determination result is a determination result indicating presence or absence of a disease: specify a region in which, among the respective weighted elements of the feature vector, a predetermined number of top elements with highest weighted values are obtained as the influential region in a case in which the determination result indicating the presence of the disease is derived; and
specify a region in which, among the respective weighted elements of the feature vector, a predetermined number of bottom elements with lowest weighted values are obtained as the influential region in a case in which the determination result indicating the absence of the disease is derived.

In addition, in the information processing apparatus according to the present disclosure, the processor may be configured to, in a case in which the determination result is a determination result indicating presence or absence of a disease: specify a region in which, among the respective weighted elements of the feature vector, an element with a weighted value equal to or greater than a first threshold value is obtained as the influential region in a case in which the determination result indicating the presence of the disease is derived; and
specify a region in which, among the respective weighted elements of the feature vector, an element with a weighted value equal to or less than a second threshold value is obtained as the influential region in a case in which the determination result indicating the absence of the disease is derived.

In addition, in the information processing apparatus according to the present disclosure, the feature of each of the second regions for each of the first regions may be a ratio of each of the plurality of second regions included in each of the first regions to the first region.

In addition, in the information processing apparatus according to the present disclosure, the feature vector may further include, as the element, a feature amount that represents at least one of a ratio of each of the plurality of second regions to a region of the target object, a ratio of each of the plurality of second regions included in each of the plurality of first regions, or a ratio of a boundary of a specific property in the region of the target object to the second region representing the specific property.

According to the present disclosure, there is provided an information processing method comprising:
dividing a target image into a plurality of first regions through a first division;
dividing the target image into a plurality of second regions through a second division different from the first division;
deriving a feature vector that represents at least a feature of each of the second regions for each of the first regions;
deriving a determination result for a target object included in the target image based on the feature vector;
specifying, among elements of the feature vector, an influential element that affects the determination result; and
specifying an influential region that affects the determination result in the target image based on the influential element.

A program causing a computer to execute the information processing method according to the present disclosure may also be provided.

According to the present disclosure, it is possible to specify a region that affects the determination result for the target object.

### BRIEF DESCRIPTION OF THE DRAWINGS

FIG. 1 is a diagram showing a schematic configuration of a diagnostic support system to which an information processing apparatus according to an embodiment of the present disclosure is applied.

FIG. 2 is a diagram showing a schematic configuration of the information processing apparatus according to the present embodiment.

FIG. 3 is a functional configuration diagram of the information processing apparatus according to the present embodiment.

FIGS. 4A and 4B are diagrams showing division results by a first division.

FIG. 5 is a diagram showing a property score corresponding to a type of property for a certain pixel.

FIG. 6 is a diagram showing a classification result by a second division.

FIG. 7 is a diagram illustrating derivation of a third feature amount.

FIG. 8 is a diagram illustrating derivation of a fourth feature amount.

FIG. 9 is a diagram showing a display screen of a target image.

FIG. 10 is a flowchart showing processing performed in the present embodiment.

### DETAILED DESCRIPTION

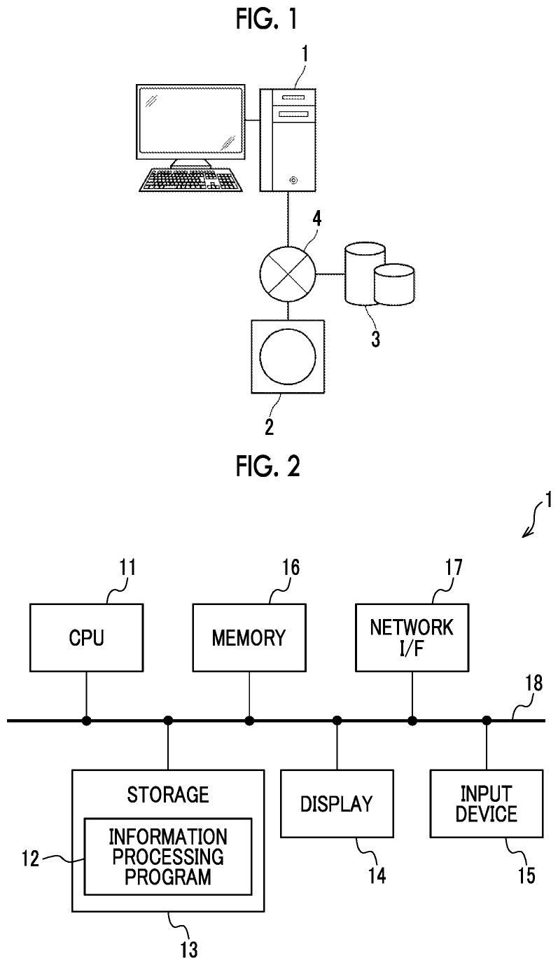

Hereinafter, an embodiment of the present disclosure will be described with reference to the drawings. FIG. 1 is a hardware configuration diagram showing an outline of a diagnostic support system to which an information processing apparatus according to the embodiment of the present disclosure is applied. As shown in FIG. 1, in the diagnostic support system, an information processing apparatus 1 according to the present embodiment, an imaging device 2, and an image storage server 3 are communicably connected to each other through a network 4.

The imaging device 2 is a device that images a site as a diagnosis target of a subject to generate a three-dimensional image showing the site and, specifically, is a CT device, an MRI device, a positron emission tomography (PET) device, or the like. The three-dimensional image consisting of a plurality of slice images, which is generated by the imaging device 2, is transmitted to and stored in the image storage server 3. In the present embodiment, the diagnosis target site of the patient who is the subject is lungs, and the imaging device 2 is a CT device and generates a CT image of the chest part including the lungs of the subject as a three-dimensional image.

The image storage server 3 is a computer that stores and manages various types of data and comprises a large-capacity external storage device and software for database management. The image storage server 3 communicates with other devices via the wired or wireless network 4 to transmit and receive image data and the like. Specifically, the image storage server 3 acquires various types of data including image data of a medical image generated by the imaging device 2 through the network, and stores the various types of data on a recording medium, such as a large-capacity external storage device, and manages the various types of data. The storage format of the image data and the communication between devices through the network 4 are based on a protocol, such as digital imaging and communication in medicine (DICOM).

Next, the information processing apparatus according to the present embodiment will be described. FIG. 2 illustrates a hardware configuration of the information processing apparatus according to the present embodiment. As shown in FIG. 2, the information processing apparatus 1 includes a central processing unit (CPU) 11, a non-volatile storage 13, and a memory 16 serving as a temporary storage area. In addition, the information processing apparatus 1 includes a display 14, such as a liquid crystal display, an input device 15, such as a keyboard and a mouse, and a network interface (I/F) 17 connected to the network 4. The CPU 11, the storage 13, the display 14, the input device 15, the memory 16, and the network I/F 17 are connected to a bus 18. The CPU 11 is an example of the processor in the present disclosure.

The storage 13 is realized by a hard disk drive (HDD), a solid state drive (SSD), a flash memory, or the like. An information processing program is stored in the storage 13 serving as a storage medium. The CPU 11 reads out an information processing program 12 from the storage 13 and then deploys the read-out information processing program 12 into the memory 16, and executes the deployed information processing program 12.

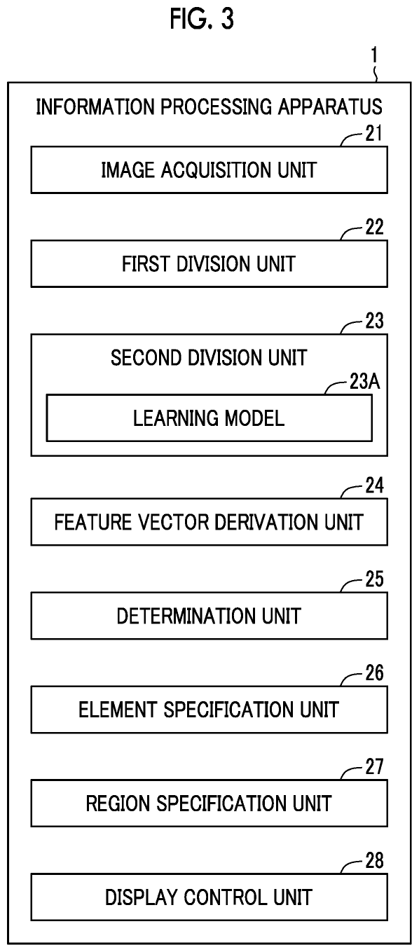

Next, a functional configuration of the information processing apparatus according to the present embodiment will be described. FIG. 3 is a diagram showing a functional configuration of the information processing apparatus according to the present embodiment. As shown in FIG. 3, the information processing apparatus 1 comprises an image acquisition unit 21, a first division unit 22, a second division unit 23, a feature vector derivation unit 24, a determination unit 25, an element specification unit 26, a region specification unit 27, and a display control unit 28. Then, the CPU 11 executes the information processing program 12, whereby the CPU 11 functions as the image acquisition unit 21, the first division unit 22, the second division unit 23, the feature vector derivation unit 24, the determination unit 25, the element specification unit 26, the region specification unit 27, and the display control unit 28.

The image acquisition unit 21 acquires a target image as an interpretation target from the image storage server 3 in response to an instruction from an interpretation physician, who is an operator, via the input device 15.

The first division unit 22 divides a target object included in the target image, that is, an anatomical structure, into a plurality of first regions through a first division. In the present embodiment, the first division unit 22 divides the lungs included in the target image into the plurality of first regions. For this purpose, the first division unit 22 extracts a lung region from the target image. As a method for extracting the lung region, any method can be used such as a method of extracting the lung by histogramming a signal value for each pixel in the target image and performing threshold processing or a region growing method based on seed points that represent the lungs. The lung region may be extracted from the target image using a discriminator that has performed machine learning so as to extract the lung region.

In the present embodiment, the first division unit 22 divides the left and right lung regions extracted from the target image based on the geometrical characteristics of the lung regions. Specifically, the lung region is divided into three first regions, that is, upper, middle, and lower regions (vertical division). As the method of vertical division, any method can be used, such as a method based on the branching position of the bronchus or a method of dividing the length or volume of the lung region in the vertical direction into three equal parts in the vertical direction. Further, the first division unit 22 divides the lung region into an outer region and an inner region (inner-outer division). Specifically, the first division unit 22 divides the left and right lung regions into lung regions, that is, an outer region that accounts for 50% to 60% of the volume of the lung region from the pleura and an inner region other than the outer region.

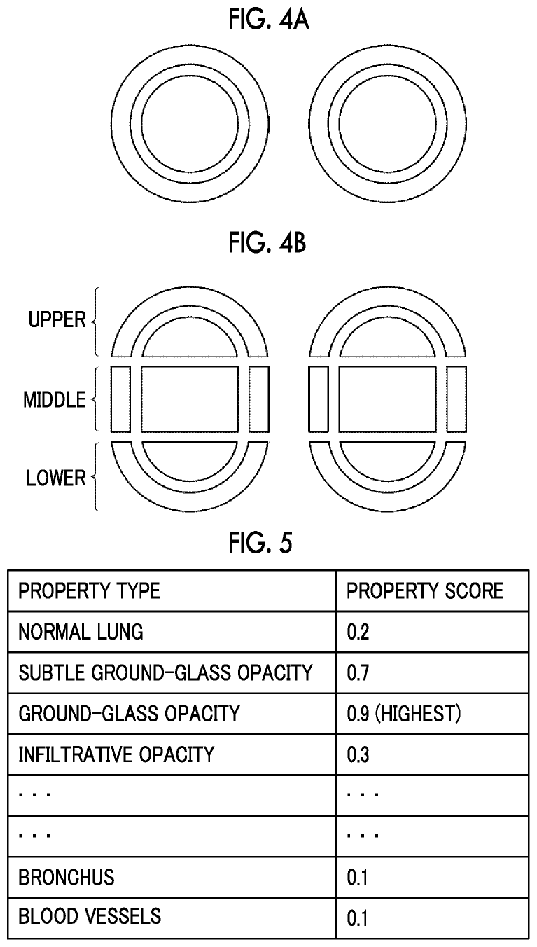

FIGS. 4A and 4B are each a diagram schematically showing the division result of the lung region. FIG. 4A shows an axial cross-section of the lung region, and FIG. 4B shows a coronal cross-section. As shown in FIGS. 4A and 4B, the first division unit 22 divides each of the left and right lung regions into six first regions.

The first division of the lung region by the first division unit 22 is not limited to the above-described method. For example, in interstitial pneumonia, which is one of lung diseases, a lesion part may spread around the bronchus and blood vessels. For this reason, a bronchial region and a vascular region may be extracted in the lung region, and the lung region may be divided into a region within a predetermined range around the bronchial region and the vascular region and a region other than the region. The predetermined range can be set as a region within a range of about 1 cm from the surfaces of the bronchus and blood vessels. In addition, the first division unit 22 may divide the lung region based on the anatomical classification of the lung region. For example, the left and right lungs may be divided into an upper lobe of the left lung, a lower lobe of the left lung, an upper lobe of the right lung, a middle lobe of the right lung, and a lower lobe of the right lung.

The second division unit 23 divides the target image into a plurality of second regions through a second division different from the first division. Specifically, by analyzing the target image, respective pixels of the lung region included in the target image are classified into a plurality of predetermined properties, and the lung region is divided into the plurality of second regions representing properties different from each other. For this purpose, the second division unit 23 includes a learning model 23A that has performed machine learning so as to discriminate the property of each pixel of the lung region included in the target image.

In the present embodiment, the learning model 23A has been trained so as to classify the lung region included in the medical image into, for example, 11 types of properties, such as normal lung, subtle ground-glass opacity, ground-glass opacity, reticular opacity, infiltrative opacity, honeycomb lung, increased lung transparency, nodular opacity, other, bronchus, and blood vessels. The types of properties are not limited to thereto, and more or fewer properties than the above properties may be used. Here, assuming that the texture of the medical image differs depending on the type of the property, the learning model 23A discriminates the property based on the texture of the medical image.

In the present embodiment, the learning model 23A consists of a convolutional neural network that has performed machine learning through deep learning or the like using training data so as to discriminate the property of each pixel of the medical image.

The training data for training the learning model 23A consists of a combination of a medical image and correct answer data representing classification results of the properties for the medical image. In a case in which the medical image is input, the learning model 23A outputs a property score for each of the plurality of properties for each pixel of the medical image. The property score is a score indicating the prominence of the property for each property. The property score takes, for example, a value of 0 or more and 1 or less, and the higher the value of the property score is, the more prominent the property is.

FIG. 5 is a diagram showing the property score corresponding to the type of property for a certain pixel. In addition, in FIG. 5, evaluation values for a part of the properties are shown for the sake of simplicity of illustration. In the present embodiment, the second division unit 23 classifies the input pixel into the property with the highest property score among the property scores for the respective properties output by the learning model 23A for the input pixel. For example, in a case in which the property scores as shown in FIG. 5 are output, the pixel is most likely to be ground-glass opacity, followed by a high probability of being subtle ground-glass opacity. On the contrary, there is almost no probability of being bronchus or blood vessels. Therefore, in a case in which the property scores as shown in FIG. 5 are output, the second division unit 23 classifies the pixel as the ground-glass opacity having the highest property score of 0.9. By performing such classification processing on all the pixels in the lung region, all the pixels in the lung region are classified into any of the plurality of types of properties. Then, the second division unit 23 divides the lung region into the plurality of second regions for each property based on the classification result of the property.

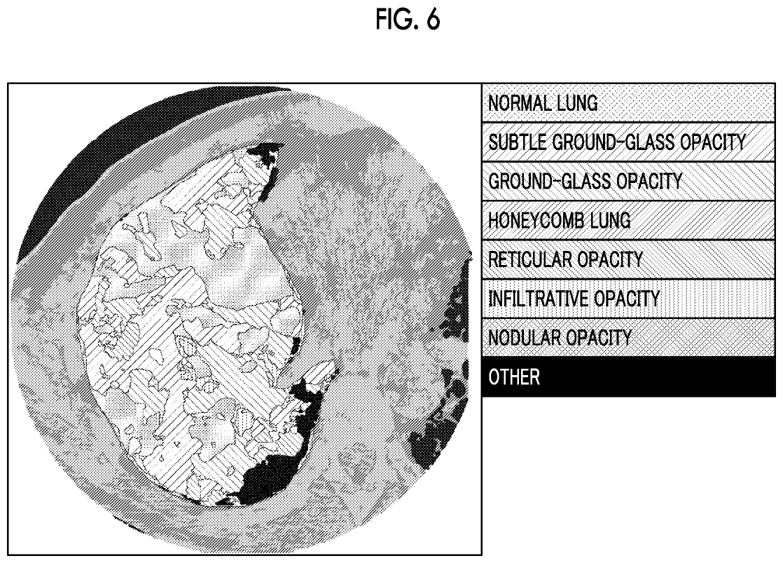

FIG. 6 is a diagram showing a division result by the second division. FIG. 6 shows a tomographic image of one tomographic plane of the target image. In addition, in FIG. 6, for the sake of simplicity of illustration, only the division results of eight types of properties, that is, normal lung, subtle ground-glass opacity, ground-glass opacity, honeycomb lung, reticular opacity, infiltrative opacity, nodular opacity, and other, are shown. A mapping image may be generated by assigning a color to the second region of each property in the target image, and the mapping image may be displayed on the display 14.

The feature vector derivation unit 24 derives a feature vector that represents at least the feature of each of the second regions for each of the first regions. In the present embodiment, the feature vector derivation unit 24 derives the feature vector including, as elements, (1) the ratio of each of the plurality of second regions included in each of the first regions to the first region (first feature amount), (2) the ratio of each of the plurality of second regions to the lung region (second feature amount), (3) the ratio of each of the plurality of second regions included in each of the plurality of first regions (third feature amount), and (4) the ratio of the area of the second regions of the properties of the subtle ground-glass opacity and the ground-glass opacity to the volume of the second regions of the properties of the subtle ground-glass opacity and the ground-glass opacity (fourth feature amount).

First, the derivation of the first feature amount will be described. The feature vector derivation unit 24 derives the volume of each of the six first regions of each of the left and right lung regions. Specifically, the number of voxels of each of the first regions is derived. In addition, for each of the first regions, the volume (that is, the number of voxels) of each of the 11 types of second regions is derived. Then, the first feature amount is derived by dividing the volume of each of the 11 types of second regions for each first region by the volume of the first region. Regarding the first feature amount, the feature vector derivation unit 24 derives the ratios of 11 volumes for the respective second regions with 11 types of properties to one first region as the first feature amounts. In the present embodiment, the left and right lung regions are each divided into six first regions. Therefore, the feature vector derivation unit 24 derives 2×6×11=132 first feature amounts.

Regarding the second feature amount, the feature vector derivation unit 24 first derives the volume of each of the left and right lung regions. In addition, the feature vector derivation unit 24 derives the volume of each of the 11 types of second regions for each of the left and right lung regions. Then, the second feature amount is derived by dividing the volume of each of the 11 types of second regions for each of the left and right lung regions by the volume of the lung region. Regarding the second feature amount, the feature vector derivation unit 24 derives the ratios of the 11 volumes for the respective second regions with the 11 types of properties to one lung region as the second feature amounts. Therefore, the feature vector derivation unit 24 derives 2×11=22 second feature amounts.

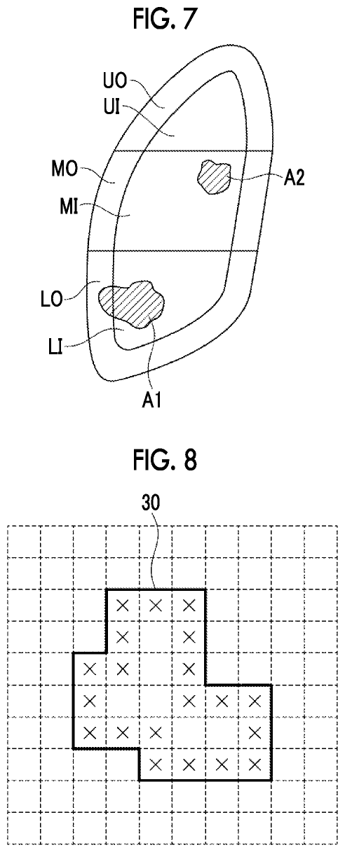

Regarding the third feature amount, the feature vector derivation unit 24 derives the volume of each of the 11 types of second regions for each of the left and right lung regions. Then, the feature vector derivation unit 24 derives, for each first region, the ratio of the derived volume of the second region included in each of the six first regions of each of the left and right lung regions. FIG. 7 is a diagram illustrating the derivation of the third feature amount. FIG. 7 shows only the right lung for the sake of illustration. As shown in FIG. 7, the right lung is divided into six first regions UO, UI, MO, MI, LO, and LI by the first division, and regions A1 and A2 of the ground-glass opacity are distributed as shown in FIG. 7 as the second regions. The feature vector derivation unit 24 derives the respective volumes V1 and V2 of the regions A1 and A2 of the ground-glass opacity and derives the total volume V0 (=V1+V2) of the regions A1 and A2 of the ground-glass opacity.

Here, as shown in FIG. 7, the region A1 of the ground-glass opacity is included in the first regions LO and LI, and the region A2 of the ground-glass opacity is included in the first region MI. Therefore, the feature vector derivation unit 24 derives volumes V11 and V12 included in the respective first regions LO and LI in the region A1 of the ground-glass opacity. Then, by dividing the volume V11 of the region A1 of the ground-glass opacity included in the first region LO by the total volume V0 of the regions A1 and A2 of the ground-glass opacity, the ratio (V11/V0) of the region of the ground-glass opacity included in the first region LO is derived as the third feature amount for the first region LO.

Similarly, by dividing the volume V12 of the region A1 of the ground-glass opacity included in the first region LI by the total volume V0 of the regions A1 and A2 of the ground-glass opacity, the ratio (V12/V0) of the region of the ground-glass opacity included in the first region LI is derived as the third feature amount for the first region LI. Further, by dividing the volume V2 of the region A2 of the ground-glass opacity included in the first region MI by the total volume V0 of the regions A1 and A2 of the ground-glass opacity, the ratio (V2/V0) of the region of the ground-glass opacity included in the first region MI is derived as the third feature amount for the first region MI. Meanwhile, the first regions UO, UI, and MO do not include the region of the ground-glass opacity. Therefore, the ratio of the region of the ground-glass opacity included in the first regions UO, UI, and MO is zero.

Regarding the third feature amount, the feature vector derivation unit 24 derives, for each of the left and right lung regions, the ratio of each of the 11 types of second regions included in each of the six first regions. Therefore, the feature vector derivation unit 24 derives 11×6×2=132 third feature amounts by combining the left and right lung regions.

Regarding the fourth feature amount, the feature vector derivation unit 24 derives the volume of the second regions of the subtle ground-glass opacity and the ground-glass opacity among the second regions included in the left and right lung regions. The derived volume is the number of voxels PV of the regions of the subtle ground-glass opacity and the ground-glass opacity included in the left and right lung regions. In addition, the surface area of the regions of the subtle ground-glass opacity and the ground-glass opacity are derived. The derived surface area is the number of voxels PA present on the surface of the regions of the subtle ground-glass opacity and the ground-glass opacity. Then, the feature vector derivation unit 24 derives the fourth feature amount (PA/PV) by dividing the number of voxels PA by the number of voxels PV.

FIG. 8 is a diagram illustrating the derivation of the fourth feature amount. In FIG. 8, for the sake of illustration, the region of the ground-glass opacity is two-dimensionally shown. Therefore, one square in FIG. 8 represents one voxel. As shown in FIG. 8, the number of voxels PV of a region 30 of the ground-glass opacity is 26. Meanwhile, the number of voxels PA present on the surface of the region of the ground-glass opacity is 20. In FIG. 8, the voxel present on the surface of the region of the ground-glass opacity is marked with an x symbol. In this case, the feature vector derivation unit 24 derives the fourth feature amount as PA/PV, that is, 20/26.

There may be a case in which a plurality of regions of the subtle ground-glass opacity and the ground-glass opacity are present in the lung region. In this case, the feature vector derivation unit 24 derives the fourth feature amount by dividing the sum of the surface areas of all the regions of the subtle ground-glass opacity and the ground-glass opacity by the sum of the volumes. Therefore, the feature vector derivation unit 24 derives one fourth feature amount.

The feature vector derivation unit 24 derives the feature vector having each of the first to fourth feature amounts as an element. Since the first feature amount is 132, the second feature amount is 22, the third feature amount is 132, and the fourth feature amount is 1, the number of derived elements of the feature vector is 287.

The determination unit 25 derives a determination result for the lung region, specifically, a determination result indicating the presence or absence of a disease, based on the feature vector derived by the feature vector derivation unit 24. For example, it is assumed that the determination unit 25 derives a determination result indicating the presence or absence of coronavirus pneumonia due to the new coronavirus disease.

Here, in the present embodiment, the determination unit 25 derives the determination result indicating the presence or absence of coronavirus pneumonia in the lung region by linearly discriminating each element of the feature vector. Specifically, the determination unit 25 consists of a discriminator that calculates a weighted addition value S0 of each element of the feature vector by Equation (1) and that outputs the determination result indicating coronavirus pneumonia in a case in which the calculated weighted addition value S0 is equal to or greater than a threshold value Th0 and that outputs the determination result indicating non-coronavirus pneumonia in a case in which the calculated weighted addition value S0 is less than the threshold value Th0. In Equation (1), αk is an element of the feature vector, and mk is a weight of the element αk of the feature vector. k corresponds to the element of the feature vector (k=1 to 287)

S0=Σ(mk×αk) (1)

Here, the weight coefficient mk in Equation (1) will be described. In the present embodiment, the weight coefficient mk is decided by machine learning. For machine learning, in the present embodiment, a plurality of pieces of positive training data consisting of a combination of a feature vector derived from a medical image known to be coronavirus pneumonia (hereinafter referred to as a coronavirus medical image) and the weighted addition value S0 calculated from the feature vector are prepared. In addition, a plurality of pieces of negative training data consisting of a combination of a feature vector derived from a medical image known to be non-coronavirus pneumonia (hereinafter referred to as a non-coronavirus medical image) and the weighted addition value S0 calculated from the feature vector are prepared. Then, machine learning is performed in which the weight coefficient mk is decided such that the weighted addition value S0 is equal to or greater than the threshold value Th0 in a case in which the positive training data is used and the weighted addition value S0 is less than the threshold value Th0 in a case in which the negative training data is used, whereby the discriminator is constructed.

The acquisition of the determination result performed by the determination unit 25 is not limited to the above linear discrimination. For example, the discriminator may consist of a neural network such as a support vector machine (SVM) or a convolutional neural network (CNN).

The element specification unit 26 specifies, among the elements of the feature vector, an influential element that affects the determination result indicating the presence or absence of coronavirus pneumonia. In the present embodiment, the determination result indicating the presence or absence of coronavirus pneumonia is derived by performing the linear discrimination in the determination unit 25. In a case in which a determination result indicating the presence of coronavirus pneumonia is derived, the element specification unit 26 compares the values of the weighted elements mk×αk in Equation (1) for all the elements of the feature vector and specifies a predetermined number of top elements with highest values of mk×αk as the influential elements. For example, the element specification unit 26 specifies the top three elements with the highest values of mk×αk as the influential elements. In a case in which it is assumed that the total number of elements of the feature vector is 10, 10 weighted elements of m1×α1 to m10×α10 are obtained. Among these, in a case in which the top three weighted elements with the highest values are m1×α1, m3×α3, and m7×α7, the element specification unit 26 specifies α1, α3, and α7 as the influential elements.

On the other hand, in a case in which the determination result indicating the absence of coronavirus pneumonia is derived, the element specification unit 26 compares the values of the weighted elements mk×αk in Equation (1) for all the elements of the feature vector and specifies a predetermined number of bottom elements with lowest values of mk×αk as the influential elements.

In a case in which the determination result indicating the presence of coronavirus pneumonia is derived, the element specification unit 26 may specify all the elements in which the value of mk×αk is equal to or greater than a first threshold value Th1 as the influential elements. In this case, in a case in which the determination result indicating the absence of coronavirus pneumonia is derived, the element specification unit 26 may specify all the elements in which the value of mk×αk is equal to or less than a second threshold value Th2 as the influential elements.

The region specification unit 27 specifies an influential region that affects the determination result in the lung region based on the influential element specified by the element specification unit 26. In the present embodiment, the region specification unit 27 specifies which of the first to fourth feature amounts each of all the influential elements specified by the element specification unit 26 is.

Here, in a case in which the influential element includes the first feature amount, the first feature amount is the ratio of each of the plurality of second regions included in each of the first regions to the first region. Therefore, the region specification unit 27 specifies the first region from which the first feature amount to be the influential element is derived as the influential region. For example, in a case in which the influential element is derived in the upper and outer first region of the first regions of the left lung, the region specification unit 27 specifies the upper and outer first region of the left lung as the influential region. In a case in which all the influential elements are the first feature amounts, all the first regions from which the first feature amounts to be the influential elements are derived are specified as the influential regions.

On the other hand, in a case in which the influential element includes the second feature amount, the second feature amount is the ratio of each region of the plurality of types of properties to the lung region. Therefore, the region specification unit 27 specifies the entire region of the lung region from which the second feature amount is derived as the influential region. Alternatively, since 11 second feature amounts are derived in each of the left and right lung regions, the second region from which the second feature amount to be the influential element is derived may be specified as the influential region. For example, it is assumed that a plurality of specified influential elements are the second feature amounts, which are second feature amounts for a second region for the property of the honeycomb lung included in the left lung region, a second region for the property of the ground-glass opacity, and a second region for the property of the infiltrative opacity of the right lung region, respectively. In this case, the region specification unit 27 may specify the second region for the property of the honeycomb lung in the left lung region, the second region for the property of the ground-glass opacity in the left lung region, and the second region for the property of the infiltrative opacity of the right lung region, as the influential regions.

In a case in which the influential element includes only the second feature amount, whether to specify the entire regions of the above lung region as the influential regions or specify any of the second regions as the influential elements included in the lung region as the influential region need only be configured to be set from an instruction via the input device 15.

In addition, in a case in which the influential element includes the third feature amount, the third feature amount is the ratio of each of the plurality of second regions included in each of the plurality of first regions. Therefore, the region specification unit 27 need only specify the first region from which the third feature amount to be the influential element is derived as the influential region. For example, in a case in which the first region from which the influential element is derived is the inner first region on the lower side of the right lung, the first region need only be specified as the influential region.

In addition, in a case in which the influential element includes the fourth feature amount, the fourth feature amount is the ratio of the area of the second regions of the properties of the subtle ground-glass opacity and the ground-glass opacity to the volume of the second regions of the properties of the subtle ground-glass opacity and the ground-glass opacity. Therefore, the region specification unit 27 need only specify the second regions which have the properties of the subtle ground-glass opacity and the ground-glass opacity, as the influential regions.

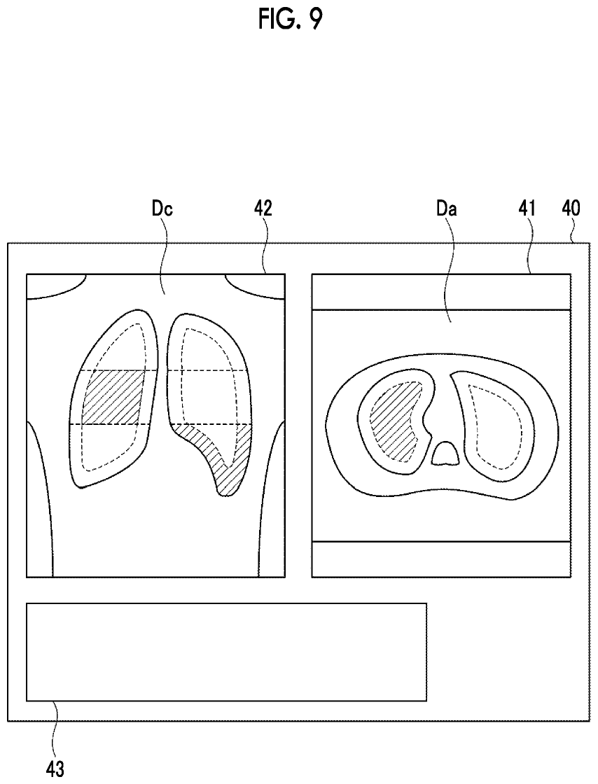

The display control unit 28 enhances the influential region specified by the region specification unit to display the target image on the display 14. FIG. 9 is a diagram showing a display screen of the target image. As shown in FIG. 9, the display screen 40 of the target image includes a first image region 41, a second image region 42, and a text region 43. A tomographic image Da of an axial cross-section of the target image is displayed in the first image region 41. A tomographic image Dc of a coronal cross-section of the target image is displayed in the second image region 42. In the tomographic images Da and Dc, broken lines are displayed at the boundary between the first regions. Further, the influential region is enhanced and displayed in the tomographic images Da and Dc. In FIG. 9, the influential region is enhanced and displayed with hatching, but the present disclosure is not limited thereto. The influential region may be enhanced and displayed by thickening the line surrounding the influential region, increasing the brightness of the influential region, or coloring the influential region.

The tomographic images Dc and Da to be displayed can be switched by moving the mouse cursor to the first image region 41 and the second image region 42 and rotating the mouse wheel. In addition, observation sentences representing an interpretation result for the tomographic images Da and Dc can be input to the text region 43.

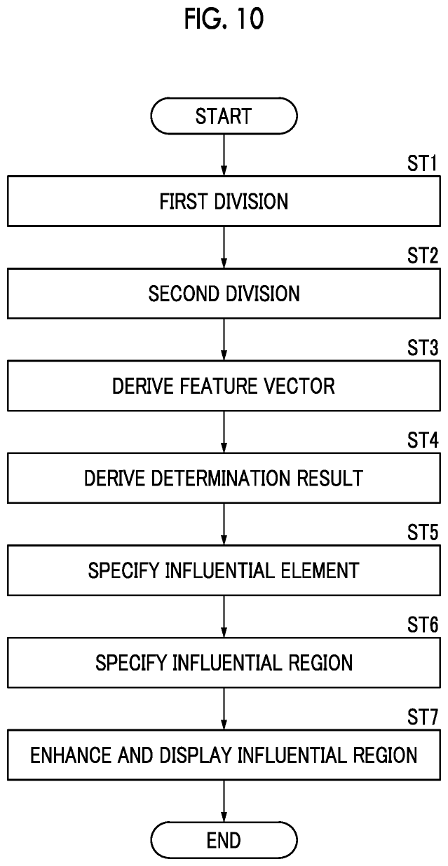

Next, processing performed in the present embodiment will be described. FIG. 10 is a flowchart showing the processing performed in the present embodiment. It is assumed that the target image as a processing target is acquired by the image acquisition unit 21 and stored in the storage 13. First, the first division unit 22 divides the lung region included in the target image into the plurality of first regions (first division; step ST1). Next, the second division unit 23 divides the lung region included in the target image into the plurality of second regions through the second division different from the first division (second division; step ST2). Then, the feature vector derivation unit 24 derives the feature vector that represents at least the feature of each of the second regions for each of the first regions (step ST3).

Subsequently, the determination unit 25 derives the determination result for the lung region, specifically, the determination result indicating the presence or absence of a disease in the lung region, based on the feature vector derived by the feature vector derivation unit 24 (step ST4). Next, the element specification unit 26 specifies the influential element that affects the determination result indicating the presence or absence of coronavirus pneumonia among the elements of the feature vector (step ST5), and the region specification unit 27 specifies the influential region that affects the determination result in the lung region based on the influential element specified by the element specification unit 26 (step ST6). Then, the display control unit 28 enhances the influential region specified by the region specification unit to display the target image on the display 14 (step ST7), and the process ends.

As described above, in the present embodiment, the feature vector representing at least the feature of each of the second regions for each of the first regions is derived, and the determination result indicating the presence or absence of the disease in the lung region is derived based on the feature vector. Further, among the elements of the feature vector, the influential element that affects the determination result is specified, and the influential region that affects the determination result in the lung region is specified based on the influential element. Therefore, according to the present embodiment, it is possible to specify the influential region within the lung region that affects the determination result regarding the presence or absence of lung diseases such as coronavirus pneumonia.

In addition, by enhancing the influential region to display the target image, the influential region included in the target image can be easily confirmed.

In the above embodiment, the feature vector derivation unit 24 derives the first to fourth feature amounts, but the present disclosure is not limited thereto. By deriving only the first feature amount, a feature vector consisting of only the first feature amount may be derived. Alternatively, by deriving only the third feature amount, a feature vector consisting of only the third feature amount may be derived. Alternatively, by deriving only the fourth feature amount, a feature vector consisting of only the fourth feature amount may be derived. A feature vector consisting of only the second feature amount in which only the second feature amount is derived may be derived. However, in this case, the second region from which the second feature amount to be the influential element is derived need only be specified as the influential region without specifying the entire regions of the lung region as the influential regions.

In addition, in the above embodiment, the lungs are used as the target object included in the target image, but the target object is not limited to the lungs. In addition to the lungs, any site of the human body such as the heart, the liver, the brain, and the limbs can be used as the target object.

Further, in the above embodiment, for example, as the hardware structure of a processing unit that executes various types of processing, such as the image acquisition unit 21, the first division unit 22, the second division unit 23, the feature vector derivation unit 24, the determination unit 25, the element specification unit 26, the region specification unit 27, and the display control unit 28, various processors shown below can be used. The above various processors include, as described above, in addition to the CPU which is a general-purpose processor that executes software (programs) to function as various processing units, a programmable logic device (PLD) which is a processor having a changeable circuit configuration after manufacture, such as a field programmable gate array (FPGA), a dedicated electrical circuit which is a processor having a dedicated circuit configuration designed to execute specific processing, such as an application specific integrated circuit (ASIC), and the like.

One processing unit may be composed of one of these various processors or a combination of two or more processors of the same type or different types (for example, a combination of a plurality of FPGAs or a combination of a CPU and an FPGA). Alternatively, a plurality of processing units may be composed of one processor.

A first example of the configuration in which the plurality of processing units are composed of one processor is an aspect in which one or more CPUs and software are combined to constitute one processor and the processor functions as a plurality of processing units, as typified by a computer such as a client and a server. A second example is an aspect in which a processor that realizes functions of an entire system including a plurality of processing units with one integrated circuit (IC) chip is used, as typified by a system on chip (SoC) or the like. As described above, various processing units are composed of one or more of the above various processors as the hardware structure.

Furthermore, as the hardware structure of the various processors, more specifically, an electrical circuit (circuitry) in which circuit elements, such as semiconductor elements, are combined can be used.

What is claimed is:

1. An information processing apparatus comprising:
at least one processor,
wherein the processor is configured to:
divide a target image into a plurality of first regions through a first division;
divide the target image into a plurality of second regions through a second division different from the first division;
derive a feature vector that represents at least a feature of each of the second regions for each of the first regions;
derive a determination result for a target object included in the target image based on the feature vector;
specify, among elements of the feature vector, an influential element that affects the determination result; and
specify an influential region that affects the determination result in the target image based on the influential element.

2. The information processing apparatus according to claim 1,
wherein the processor is configured to enhance the influential region to display the target image.

3. The information processing apparatus according to claim 1,
wherein the influential region is at least one of a region of the target object, at least one region of the plurality of first regions, or at least one region of the plurality of second regions.

4. The information processing apparatus according to claim 1,
wherein the target image is a medical image, the target object is an anatomical structure, and the determination result is a determination result regarding presence or absence of a disease.

5. The information processing apparatus according to claim 4,
wherein the first division is a division based on a geometrical characteristic or an anatomical classification of the anatomical structure, and
the second division is a division based on a property of the anatomical structure.

6. The information processing apparatus according to claim 1,
wherein the processor is configured to acquire the determination result for the target object by linearly discriminating each element of the feature vector.

7. The information processing apparatus according to claim 6,
wherein the processor is configured to perform the linear discrimination by comparing a weighted addition value of each element of the feature vector with a threshold value.

8. The information processing apparatus according to claim 7,
wherein the processor is configured to, in a case in which the determination result is a determination result indicating presence or absence of a disease: specify a region in which, among the respective weighted elements of the feature vector, a predetermined number of top elements with highest weighted values are obtained as the influential region in a case in which the determination result indicating the presence of the disease is derived; and
specify a region in which, among the respective weighted elements of the feature vector, a predetermined number of bottom elements with lowest weighted values are obtained as the influential region in a case in which the determination result indicating the absence of the disease is derived.

9. The information processing apparatus according to claim 7,
wherein the processor is configured to, in a case in which the determination result is a determination result indicating presence or absence of a disease: specify a region in which, among the respective weighted elements of the feature vector, an element with a weighted value equal to or greater than a first threshold value is obtained as the influential region in a case in which the determination result indicating the presence of the disease is derived; and
specify a region in which, among the respective weighted elements of the feature vector, an element with a weighted value equal to or less than a second threshold value is obtained as the influential region in a case in which the determination result indicating the absence of the disease is derived.

10. The information processing apparatus according to claim 1,
wherein the feature of each of the second regions for each of the first regions is a ratio of each of the plurality of second regions included in each of the first regions to the first region.

11. The information processing apparatus according to claim 1,
wherein the feature vector further includes, as the element, a feature amount that represents at least one of a ratio of each of the plurality of second regions to a region of the target object, a ratio of each of the plurality of second regions included in each of the plurality of first regions, or a ratio of a boundary of a specific property in the region of the target object to the second region representing the specific property.

12. An information processing method comprising:
dividing a target image into a plurality of first regions through a first division;
dividing the target image into a plurality of second regions through a second division different from the first division;
deriving a feature vector that represents at least a feature of each of the second regions for each of the first regions;
deriving a determination result for a target object included in the target image based on the feature vector;
specifying, among elements of the feature vector, an influential element that affects the determination result; and
specifying an influential region that affects the determination result in the target image based on the influential element.

13. A non-transitory computer-readable storage medium that stores an information processing program causing a computer to execute:
a procedure of dividing a target image into a plurality of first regions through a first division;
a procedure of dividing the target image into a plurality of second regions through a second division different from the first division;
a procedure of deriving a feature vector that represents at least a feature of each of the second regions for each of the first regions;
a procedure of deriving a determination result for a target object included in the target image based on the feature vector;
a procedure of specifying, among elements of the feature vector, an influential element that affects the determination result; and
a procedure of specifying an influential region that affects the determination result in the target image based on the influential element.
